(19) United States
(12) Patent Application Publication
(10) Pub. No.: US 2003/0088315 A1
(43) Pub. Date: May 8, 2003

(54) Patella replacement apparatus

(76) Inventor: Robert S. Supinski, Mount Morris, NY (US)

Correspondence Address: Lynn J. Alstadt, Buchanan Ingersoll, P.C., One Oxford Center, 301 Grant Street, 20th Floor, Pittsburgh, PA, 15219, US

(21) Appl. No.: 10/007,812

(22) Filed: Nov. 8, 2001

(51) Int. Cl. A61F 2/38

(52) U.S. Cl. 623/20.2

(57) ABSTRACT

A patella replacement device has a generally hemispherical member which is surrounded by a collar or metallic ring and has apertures. A second hemispherical member has projections which fit into the apertures of the first member and the combination is used for the repair or replacement of the patella bone. At least one member is made of a high porosity metal material or contains a porous coating for initial stability through the adhesive and frictional properties of the metal material or coating.

### FIELD OF INVENTION

This invention relates to a patella replacement apparatus and more particularly to an improved patella prosthesis allowing improved biological fixation to the patella knee area of a patient user.

### BACKGROUND OF THE INVENTION

Various prosthetic devices for replacement of the articulating surface of the patella or knee cap have been designed for implantation in cases where replacement of the natural articulating surface of the patella is indicated. Generally, the posterior surface of the patella is resected and replaced by an artificial articulating surface. These devices have been designed for implantation in many cases where replacement of the articulating surface of the patella is indicated. There are many examples of such devices in the prior art and there are many devices which are commercially available to surgeons. Examples of such devices are disclosed in U.S. Pat. No. 5,019,104 to Whiteside, et al., U.S. Pat. No. 4,007,495 to Frazier and U.S. Pat. No. 5,314,489 to Elloy et al. One common patella replacement device has two basic components, a metal portion that is attached opposite the femur and a plastic portion that fits onto the metal portion. Frazier discloses a patella femoral prosthesis having a patella prosthesis and a femoral prosthesis which are connected to each other. The femoral prosthesis is formed of steel and is attached onto the femur by bone cement. The patella prosthesis is formed of polyethylene or a similar plastic material and is mounted on the patella. The two prostheses are connected together through a pin and slot arrangement. The device disclosed by Elloy et al. in U.S. Pat. No. 5,314,480 has a femoral component and a tibial component including a metal disk imbedded within a high molecular polyethylene disk. The polyethylene disk surrounds the edge of the metal disk so that the metal disk is flush with the polyethylene disk on one surface. The metal disk is provided with serrations which fit into the polyethylene disk to prevent rotation of the metal disk relative to the polyethylene disk.

Patella replacement devices must be able to fix biologically to remnant bone or soft tissue such as muscle or tendons. Additionally, the devices must articulate and function within the confines of the cartilage on the femoral bone. The survivorship rate of devices that attempted to replace the patella has been poor and surgeons often choose to remove the total patellar bone, leaving the patient with a compromised knee joint. Removal of the patellar bone creates about a 20% loss of the quadriceps mechanism muscle strength. Research has concluded that many patients only regain about 75% of their knee normal strength. For the case of removal of a failed prosthetic patellar component from a total knee joint replacement procedure, the amount of cancellous bone remaining is minimal and offers the surgeon a challenge for the new implant stability and long term fixation, while attempting to provide biomechancial articulation against the existing femoral component. The incidence of pain, lack of full function, and rate of component loosening has been unacceptably high.

It is therefore an object of the present invention to provide an improved patella replacement device that can restore or mimic the biomedical function of the normal patella bone. In this manner, the replacement by the patella replacement device of this invention results in an improved operation and improved muscle use for the patient. The device is simple to install as compared to prior art devices and offers advantages both to the surgeon and to the patient.

### SUMMARY OF THE INVENTION

A patella replacement device for use in repairing or replacing the destroyed natural patella of a patient has two generally hemispherical members. One of the two members has a porous surface and is attached to the other member during implantation. The second member need not have a porous surface and is attached to the first member with bone cement or by mechanical means. Both members are fabricated from biocompatible materials. One member preferably is a plastic and the other member preferably is metal and has a porous surface. The porous surface allows biological fixation of the device to the patella region of the patient. The first member has a rounded fixation surface for implantation within the patella region of a patient and a relatively flat surface opposite the rounded surface. The second member has a top rounded surface and an opposing surface. At least one projection extends from a flat surface in one member into a mating aperture in the flat surface of the other member to enable the first member to couple to the second member with the second member operative to allow articulation against the femoral area of the patient. A ring having a greater diameter than the diameters of the first and second members and suture holes about the periphery of the ring can be positioned between that first member and the second member. Alternatively, an annular collar containing suture holes can be provided on either or both of the first member and the second member. Both members can be made of a ceramic or other biocompatible material rather than one component being metal and the second component being plastic.

An alternative embodiment of the patella replacement apparatus also has two members similar in shape to those in the first embodiment, but having no collar. Suture holes are preferably provided through the circumference of the second member. This embodiment does not have an annular shoulder or intermediate ring.

In yet another embodiment the metal member or ceramic member is not itself porous, but is coated with a plasma spray hydroxyapatite coating. That coating is porous.

### BRIEF DESCRIPTION OF THE FIGURES

FIG. 1 is a side view of a first embodiment of a patella replacement device according to the present invention and shows the two members of the device connected together.

FIG. 2 is an exploded view of the embodiment shown in FIG. 1.

FIG. 3 is a plan view of the flat surface of one member of the embodiment of FIG. 1.

FIG. 4 is a plan view of the flat surface of the second member of the embodiment of FIG. 1.

FIG. 5 is an exploded view similar to FIG. 2 of a second embodiment of the invention utilizing a patella ring.

FIG. 6 is a plan view of the ring in the embodiment shown in FIG. 5.

FIG. 7 is a plan view of the assembled ring and patella insert shown in FIG. 5.

FIG. 8 is an exploded view of a third embodiment of the patella replacement apparatus of the present invention.

FIG. 9 is a cross-sectional view taken along the line IX-IX of FIG. 8.

### DETAILED DESCRIPTION OF THE PREFERRED EMBODIMENTS

Referring to FIGS. 1 through 4 there is shown a present preferred embodiment of the patella knee replacement device 10 which consists of two members or components 11 and 12. One member 11 is a bearing component and has a rounded top surface and a flat bottom surface giving the component a generally hemispherical shape. The flat bottom surface may be circular or elliptical depending upon the patient for whom the device is used. The second member 12 is similar in shape to the first component. The first and second members are made from a biocompatible material such as plastic, particularly polyethylene, biocompatible metals, such as titanium or cobalt chrome alloys, or a ceramic. I prefer to make one component from a metal and the second component from a plastic. The projections preferably are plastic. At least a portion of the second component 12 has a porous surface. The surface can be formed through use of a porous metal, by roughening the surface, by providing a beaded surface or other conventional means. In the first embodiment both components 11 and 12 have an annular collar 13 or 14 surrounding the flat surface of the component. There are a series of suture holes 15 provided in the collars. The edges of the suture holes should be beveled and smooth so as not to cut the sutures. When the first and second components are assembled together the suture holes in the collars will be aligned. In this embodiment the bottom surface of the polyethylene bearing component 11 has three upstanding posts 17. I prefer that the posts be plastic. The posts can extend from either the first or second component. The posts 17 are inserted into apertures 18 of the second component 12. The apertures may be holes or slots. Any number of posts and corresponding holes or slots could be provided. The posts and apertures are preferably sized and configured to provide a snap fit. Preferably a cement is applied to the mating flat surfaces. A pair of transverse holes 16 is provided in component 12 that can also be used for sutures. The porous surface in the second component 12 allows for bone or soft tissue in-growth. The surgeon actually receives as a patella replacement device the base unit 11 and the bearing component 12 separated from one another. These components are secured together during the surgical procedure by the surgeon.

All individuals are not the same size and not the same weight. Consequently, a single patella replacement device will not work for all people. In order to provide a complete range of patella replacement devices, one needs to accommodate men, women, and children of various heights and weights. The diameter and thickness of the devices will vary accordingly. Indeed, the devices may have an elliptical cross-section with the major and minor diameters being different among the various sizes of the device. For a small size, the minor diameter of the flat surface of each member may be 0.958 inches (24.3 mm) and the major diameter may be 1.099 inches (28 mm). The height or thickness of each member may vary from 0.276 inches (7 mm) to 0.709 inches (18 mm) to allow for different knee configurations. For a medium individual, the minor diameter may be 1.063 inches (27 mm), the major diameter 1.218 inches (31 mm), and the height may vary from between 0.276 to 0.799 inches (7 mm to 20 mm). For a large individual, the minor diameter may be 0.555 inches (14 mm) and the major diameter may be 1.336 inches (34 mm). For an extra large individual, the minor diameter may be 0.607 inches (15 mm) and the major diameter may be 1.454 inches (37 mm). The component height for both the large and extra large person varies between 0.276 inches (7 mm) to 0.709 inches (18 mm) which essentially is the variation in height of the size for the small individual. The position of the posts 17, holes 18 and their diameters is the same for all sizes. The replacement device has a shape to conform to the patella of a typical user. A surgeon would measure the patient's knee area and select a patella insert having suitable dimensions for that patient.

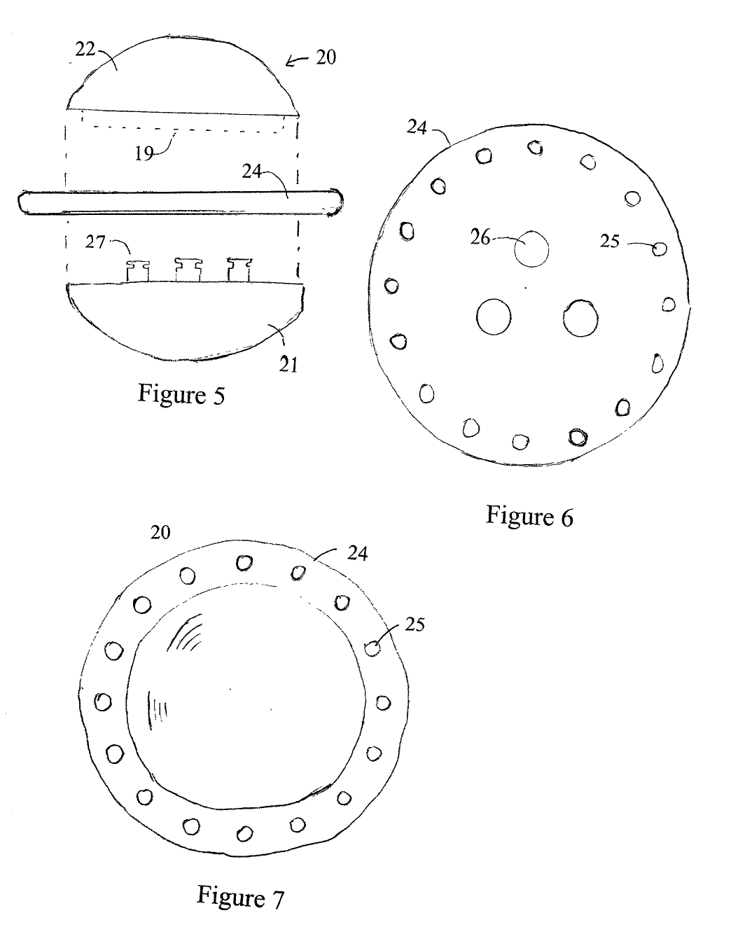

A second preferred embodiment is shown in FIGS. 5, 6 and 7. In this embodiment 20 there is a first component 21 and a second component 22 similar to the two components 11 and 12 of the first embodiment shown in FIGS. 1 through 4. However, components 21 and 22 do not have a collar. Instead there is ring 24 that is positioned between components 21 and 22. Ring 24 is can be fabricated from titanium, cobalt chrome or tantalum and contains suture holes 25 about its periphery. However, I prefer to make the ring of plastic because plastic will not cut the sutures or damage surrounding tissue as can happen with a metal ring. In this embodiment the first component 21 is polyethylene or other bio-compatible material and the second component 22 is a metal having a porous surface. Posts 27 associated with the polyethylene bearing unit 22 pass through apertures 26 in the ring 24 and are inserted into apertures (not shown) which are formed in the base unit 22. These apertures could be holes or slots.

The patella ring 24 could have a central aperture (not shown) which is of a diameter slightly less than a central flange portion (not shown) extending from member 21 or 22 similar to the collars 13 and 14 in the first embodiment. The ring could be force-fit over that region of the patella assembly by means of a press or a clamping system to provide an interference bond or fit. To provide an interference fit, the diameter of the aperture may be between one and four thousandths of an inch less than the diameter of the flange portion. The apertures 25 enable a physician to apply sutures to secure the ring and the patella insert to the knee area of a patient. Again, because people vary in height and weight, members 21 and 22 as well as the ring should be available in different sizes.

The assembly as it reaches the physician, consists of the ring 24 together with member 22 assembled as a single unit with the ring attached to or force-fitted over member 22. The polyethylene section 21 is separately supplied. All the units are ordered by the physician or the surgeon after measurements are made considering the size of the patient's knee and of course, considering the size and weight of the patient.

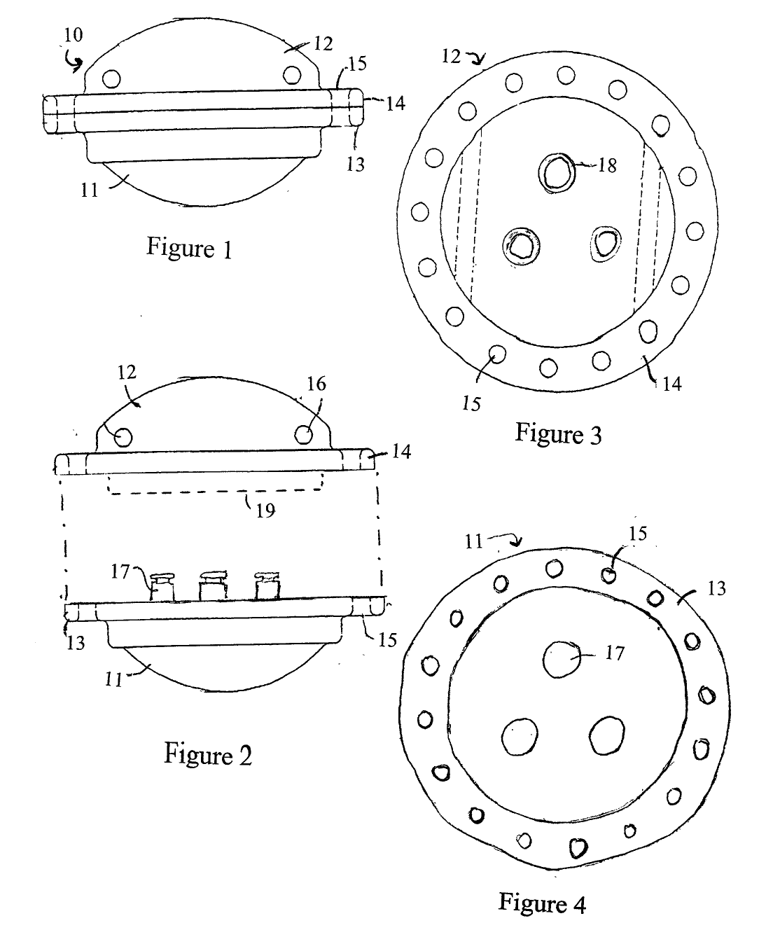

Either of the first two embodiments could be modified as shown in FIGS. 2 and 5 to provide an intermediate portion 19 on the flat surface of either component. Intermediate portion 19 has a smaller diameter than the diameter of the flat surface. Consequently, there is a peripheral gap 40 formed when the two members 11 and 12 or 21 and 22 are joined together. This peripheral gap 40 enables the surgeon to wrap soft tissue in the gap to further secure the prosthetic device and to assure optimum coupling to the knee area.

Although the second embodiment in a form containing the metal ring with a central aperture could be used, I have discovered that after such a device has been implanted, the ring can cut through the prosthesis. This can occur when movement of the patient causes the ring to move relative to the other members of the prosthesis. That movement creates a rubbing action that cuts into the prosthesis. Another problem that may occur when a metal ring is used happens when the knee is subjected to a radial force parallel with the flat surfaces of the ring. That force may cause the edge of the ring to cut surrounding soft tissue. Similar problems can occur with a plastic ring.

The smooth top surface or dome of components 12 or 22 faces outward in the knee area of the patient. The porous surface of those components 12 and 22 enables bone and/or soft tissue growth into the device promoting better security. The plastic member 11 or 21 acts as an articulator surface against the patient's cartilage or prosthetic femoral replacement enabling the patient to resume normal activities and be pain free.

The embodiments disclosed in FIGS. 1 through 7 could have a plastic component and a metal component. The second member 12 or 22 must be porous to allow bone and soft tissue to grow into the component and anchor the prosthesis. That objective cannot easily be obtained if the component is made of some metals or ceramics. Yet, I have discovered that it is possible to coat a metal or ceramic component with hydroxyapatite and thereby provide a porous structure into which bone and soft tissue can grow.

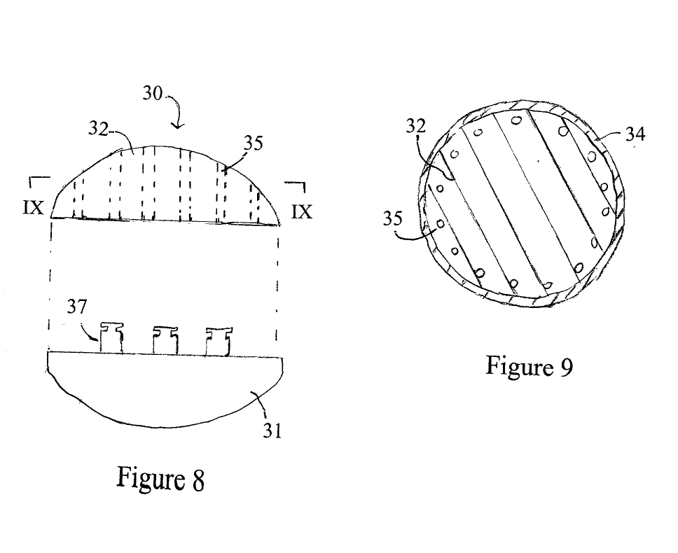

A third present preferred embodiment 30 is shown in FIGS. 8 and 9. This embodiment has two components 31 and 32 that fit together in the same way as previous embodiments. That is, posts 37 in member 31 fit into apertures (not shown) in member 32. In this embodiment, component 31 is made of a biocompatible plastic material, preferably polyethylene or a ceramic. Component 32 is metal or ceramic. A cement is applied to the flat surfaces of these components to bond them together. The second member 32 is coated with hydroxyapatite 34 to provide a porous surface that acts as a scaffold for tissue growth. After the device is implanted body fibers grow into the porous surface to secure the prosthesis. Suture holes 35 are provided around the perimeter of the second component 32 to permit the prosthesis to be initially secured in place. The sutures preferably are made of a material such as PLLA that decomposes to provide absorbable anchors. The hydroxyapatite coating is created by applying a plasma spray containing hydroxyapatite powder that bonds on impact with the surface of the plastic component. If desired, the hydroxyapatite coating could be placed on both components 31 and 32, particularly if both components are metal or ceramic. Although I prefer to coat the entire curved surface of the component, less than the entire surface could be treated with hydroxyapatite.

Those skilled in the art will recognize that hydroxyapatite is one of the several bone growth materials used by surgeons. Consequently, it should be possible to use other bone growth materials in place of or in combination with hydroxyapatite in the coating. Such other materials include human bone particles, bovine bone particles, ground coral and calcium sulfate.

The present prosthesis is implanted using standard surgical techniques. The unit will function if a patella bone is completely removed from a patient or, if patella fragments that remain attached to the soft tissue post trauma are exposed and the loose fragments are removed; or if the primary patella component that failed is removed and a remaining patella bone shell is prepared to accept the device. The sutures will initially hold the first component in place enabling the component to act as a base. After the base is stable, the posts of one component are fit into the three holes in the other component. Trial articulation against the femur with the whole knee flexing and extending will be performed and patella tracking and joint tensioning is assessed. The implantable second patella component is then cemented into place. The surgeon holds the first component against the second component until the cement is cured and hardened. The joint is reduced and the incision is closed.

I have implanted prototypes of the embodiment shown in FIGS. 1 through 5 in several patients. I also implanted in a patient a prototype of the embodiment of FIGS. 8 and 9 in which member 32 was titanium with a hydroxyapatite coating and the other member 31 was polyethylene. The patients were examined at regular intervals after surgery. The patients were able to regain nearly complete function of the knee joint and suffered no discomfort after the incision had fully healed.

Although I have shown and described certain present preferred embodiments of my patella replacement device, it should be distinctly understood that the invention is not limited thereto, but may be variously embodied within the scope of the following claims.

What is claimed is:

1. A patella replacement device for use in repairing or replacing the destroyed natural patella of a patient, comprising:
a first member fabricated from a biocompatible porous metal material and having a rounded fixation surface for implantation in the patella region of a patient with the porous metal allowing biological fixation to the patella region of the patient, said first member having a relatively flat surface opposite said rounded surface and having at least one aperture therein; and
a second member fabricated from a biocompatible joint articulating material and having a top rounded surface and an opposing surface having an extending projection for coacting with said aperture to enable said first member to couple to said second member with said second member operative to allow articulation against the femoral area of said patient.

2. The patella replacement device according to claim 1 further comprising an annular ring having a central aperture for coupling to said flat surface of said first member and having a plurality of apertures about a periphery of said ring, the periphery surrounding and extending from a peripheral edge of said first member.

3. The patella replacement device according to claim 2 wherein said annular ring is fabricated from a biocompatible metal.

4. The patella replacement device according to claim 3 wherein said metal is titanium.

5. The patella replacement device according to claim 1 wherein said second member is fabricated from polyethylene.

6. The patella replacement device according to claim 1 wherein said second member is fabricated from titanium or cobalt chrome.

7. The patella replacement device according to claim 1 also comprising at least one annular collar attached to at least one of the first member and the second member.

8. A patella replacement device for use in repairing or replacing the destroyed natural patella of a patient, comprising:
a first member fabricated from a porous metal material and having a rounded fixation surface for implantation in the patella region of a patient with said porous metal allowing biological fixation to said patella region of said patient, said first member having a relatively flat surface opposite said rounded surface and having at least one aperture therein, and
a second member fabricated from a biocompatible material and having a top rounded surface and an opposing surface having an extending projection for coacting with said aperture in said first member and dimensional so that a peripheral gap is formed between said first and second member when said projection is inserted into said aperture, said gap enabling the accommodation of soft tissue.

9. The patella replacement device according to claim 8 further comprising:
an annular ring having a central aperture for coupling to said flat surface of said first member and having a plurality of apertures about a periphery of said ring, the periphery surrounding and extending from a peripheral edge of said first member

10. The patella replacement device according to claim 9 wherein said annular ring is fabricated from a biocompatible metal.

11. The patella replacement device according to claim 10 wherein said metal is titanium.

12. The patella replacement device according to claim 8 wherein said second member is fabricated from polyethylene.

13. The patella replacement device according to claim 8 wherein said second member is fabricated from titanium or cobalt chrome.

14. The patella replacement device according to claim 8 wherein said relatively flat surface of said first member has three apertures, with said second member having three projections each adapted to coact with a respective associated one of said apertures.

15. A patella replacement device for use in repairing or replacing the destroyed natural patella comprising:
a first member fabricated from a porous metal material, said first member having a rounded fixation surface for implantation in the patella region of a patient, and a relatively flat surface opposite said rounded surface, said flat surface having at least one aperture therein;
an annular ring secured about said first member, said ring having a central aperture, an extending flange portion surrounding said first member and a plurality of apertures about a periphery thereof; and
a second member fabricated from a biocompatible material having a top round surface and an opposing surface having an extending projection for coacting with said aperture in said first member and dimensioned so that a peripheral gap is formed between said first and second members when said projection of said second member is inserted into said aperture of said first member.

16. The patella replacement device according to claim 15, wherein said annular ring is secured to said first member by an interference fit.

17. The patella replacement device according to claim 15 wherein said second member is fabricated from polyethylene.

18. The patella replacement device according to claim 15 wherein said annular ring is fabricated from titanium.

19. The patella replacement device according to claim 15 wherein said first member has three apertures on said flat surface.

20. The patella replacement device according to claim 19 wherein said second member has three projections each one operative to coact with an associated one of said three apertures of said first member.

21. The patella replacement device according to claim 15 wherein said porous metal material accommodates a bone cement placed in said at least one aperture.

22. A patella replacement device for use in repairing or replacing the destroyed natural patella of a patient comprising:
a first member fabricated from a biocompatible material and having a rounded fixation surface for implantation in the patella region of a patient, said first member having a relatively flat surface opposite said rounded surface and at least one aperture therein;
a second member fabricated from a biocompatible material and having a rounded surface and an opposing surface having an extending projection for coacting with said aperture to enable said first member to couple to said second member; and
a porous coating containing at least one bone growth material and applied to at least a portion of at least one of said first member and said second member.

23. The patella replacement device according to claim 22 wherein the at least one bone growth material is selected from the group consisting of hydroxyapatite, human bone particles, bovine bone particles, ground coral and calcium sulfate.

24. The patella replacement device according to claim 22 wherein at least one of said first member and said second member is fabricated from a material selected from the group consisting of biocompatible metals, biocompatible plastics and biocompatible ceramics.

25. The patella replacement device according to claim 22 wherein said first member is fabricated from a metal and said second member is fabricated from a plastic.

26. The patella replacement device according to claim 25 wherein said plastic is polyethylene and said metal is titanium or cobalt chrome.
